(19) United States
(12) Patent Application Publication
(10) Pub. No.: US 2011/0274775 A1
(43) Pub. Date: Nov. 10, 2011

(54) EXTRACTS OF SOUTHERNWOOD AND TOPICAL USES THEREOF

(76) Inventors: Michael Anthonavage, Lebanon, NJ (US); Meghan Russell, Ewing, NJ (US); Samantha Tucker-Samaras, Long Valley, NJ (US)

(21) Appl. No.: 12/775,938

(22) Filed: May 7, 2010

(51) Int. Cl. A61K 36/282; A61K 31/702; A61P 17/06; A61P 17/00; A61P 17/04; A61K 31/7016; A61K 31/7004

(52) U.S. Cl. 424/740; 514/53; 514/61; 514/23

(57) ABSTRACT

The present invention relates to a method of treating visibly dry skin. The method includes topically applying a composition comprising a Southernwood extract to said skin.

### FIELD OF THE INVENTION

The present invention relates to compositions comprising as well as methods of using compositions comprising extracts of Southernwood. The compositions are useful, for example for treating visibly dry skin.

### BACKGROUND OF THE INVENTION

The top layer of human skin, the stratum corneum (SC) consists of protein enriched corneocytes embedded in a lipid matrix. The major function of the structure, as the body's protective barrier to the environment that prevents the loss of water and nutrients from within, is predominantly determined by the levels, composition and structure of the SC lipids. The SC lipids also influence the mechanical and desquamatory (skin cell shedding) activities of the SC. Abnormalities in the SC lipids can occur in connection with various conditions such as aged or photo damaged skin, or in connection with xerosis (a condition of abnormal dryness) such as during winter months.

Replenishing SC lipids by topical application of hydrophobic compounds is an approach that has been used with limited success. A longer lasting approach is to exploit the robust epidermal lipid biosynthetic pathways of the viable epidermis using natural extracts or compounds that upregulate the body's natural production of these lipids, and particularly a class of critically important SC lipids known as ceramides. Nicotinamide, for example, has been reported to increase the synthesis of ceramides, major constituents of lipids present as lamellar sheets in intercellular spaces of the SC. Br. J. Dermatol. (2000 September) 143(3):524-31. In addition, certain isomers of lactic acid are also reported to stimulate ceramide biosynthesis. Arch. Dermatol. Res. (1996) 288: 383-390.

It is well recognized that ceramides are functionally and structurally distinct from lipids that are present in deeper layers (i.e., the hypodermis) of the skin. For example, while ceramides are a class of polar lipids that play a role in cell membrane structures to enhance skin barrier function, the deeper hypodermal lipids are non-polar lipids whose function relates to energy storage, thermal insulation, and protection of internal organs from mechanical injury.

It has been reported that Southernwood extract is suitable for “fat restructuring” and stimulating adipogenesis, presumably by affecting the non-polar lipids of hypodermis. See US 2009/0285868 A1. Surprisingly, the inventors have now found that Southernwood extract, acting on an entirely different biological pathway, is suitable to enhance the biosynthesis of ceramides. Accordingly, the inventors have found that Southernwood extracts are remarkably suitable for treating completely different need states, and completely different skin, than what is known in the art. Specifically, the inventors have found that Southernwood extract is surprisingly suitable for the topical treatment of visibly dry, e.g., xerotic skin.

### SUMMARY OF THE INVENTION

The present invention relates to a method treating visibly dry skin. The method includes topically applying a composition comprising a Southernwood extract to said skin.

Other features and advantages of the present invention will be apparent from the detailed description of the invention and from the claims.

### DETAILED DESCRIPTION OF PREFERRED EMBODIMENTS

Unless defined otherwise, all technical and scientific terms used herein have the same meaning as commonly understood by one of ordinary skill in the art to which the invention pertains. Also, all publications, patent applications, patents, and other references mentioned herein are incorporated by reference. Unless otherwise indicated, a percentage refers to a percentage by weight (i.e., % (W/W). Unless stated otherwise, all ranges are inclusive of the endpoints, e.g., “from 4 to 9” includes the endpoints 4 and 9.

As used herein, “visibly dry skin” refers to dry or dehydrated skin that shows visible signs of flaking. In certain embodiments of the invention, visibly dry skin is characterized clinically according to Rawlings et al., J. Soc. Cosmet. Chem. 45: 203-220 July/August 1994, in one or more of the following manners: skin having small flakes of dry skin and whitening of dermatoglyphic triangles (mild xerosis); skin having small, dry flakes giving a light powdery appearance—corners of dermatoglyphic triangles have started to uplift (moderate xerosis); or skin in which the entire length of a number of dermatoglyphic triangles have uplifted to generate large, dry skin flakes—roughness is very evident (well-defined xerosis). In certain other embodiments of the invention, the visibly dry skin has deficient levels of ceramides in the stratum corneum.

In certain embodiments, the visibly dry skin may be, but is not necessarily, classified as skin having compromised barrier properties, or skin characterized by certain disease states such as eczematic skin, psoriatic skin, or skin characterized by atopic dermatitis. As used herein, “eczema” refers to a chronic skin disorder that involves inflammation of the epidermis and often presents as scaly and itchy rashes. As used herein, “atopic dermatitis” refers to a type of chronically relapsing, non-contagious and pruritic form of eczema. As used herein, “psoriasis” refers to a chronic, non-infectious disease that presents as red, scaly patches or plaques of excessive inflammation or excessive skin production, often present on extensor surfaces such as knees and elbows.

The visibly dry skin may be present on the face or body, including the hands, feet, scalp, elbows, knees, ankles, nape of the neck, among other areas of the body.

As used herein, “treating” refers to mitigating, reducing, preventing, improving, or eliminating the presence or appearance of a condition or disease.

As used herein, “cosmetic” refers to a beautifying substance or preparation which preserves, restores, bestows, simulates, or enhances the appearance of bodily beauty or appears to enhance beauty or youthfulness, specifically as it relates to the appearance of tissue or skin.

As used herein, “cosmetically acceptable” means suitable for use in contact with (human) tissues (e.g., the skin) without undue toxicity, incompatibility, instability, irritation, allergic response, and the like, commensurate with a reasonable benefit/risk ratio.

It is believed that one skilled in the art can, based upon the description herein, utilize the present invention to its fullest extent. The following specific embodiments are to be construed as merely illustrative, and not limitative of the remainder of the disclosure in any way whatsoever.

As used herein the term “Southernwood” refers to plants of the genus/species Artemesia arbrotaum. As used herein the term “extract of Southernwood” or “Southernwood extract” refers to extract obtained from one or more parts of the

Southernwood plant (e.g., flower, seed, root, rhizome, stem, fruit and/or leaf), which may be optionally isolated. The Southernwood extract may for example be prepared by finely dividing the harvested plant or parts thereof, such as by crushing, grinding, or milling to a finely divided solid or powder. The plant or portions thereof are preferably contacted with a solvent, e.g., steam, water (that may or not be heated), or other solvents, to extract portions thereof.

In certain embodiments of the invention, the Southernwood extract comprises a mixture of oligosaccharides. In certain embodiments, the Southernwood extract comprises oligosaccharides having a degree of polymerization of 1 to 4, and polyphenols. In another embodiment, the Southernwood extract comprises oligosaccharides having a degree of polymerization of 1 to 4, proteins, and polyphenols. For example, the Southernwood extract may have an actives content (i.e., the portion that excludes all solvents employed in the extraction process) comprising about 1% polyphenols, about 16% proteins, and about 83% saccharides having a degree of polymerization of 1 to 4. The saccharides may comprise monosaccharides, trisaccharides, and tetrasaccharides. In one embodiment, the saccharides may include monosaccharides (e.g., about 54%), trisaccharides (about 12%), and tetrasaccharides (e.g., about 34%).

One particularly suitable Southernwood extract is commercially available as PULPACTYL from Silab of France, which is an aqueous-glycolic extract of Southernwood and includes water and butylene glycol.

A suitable extraction process may comprise contacting one or more parts of the Southernwood plant with a solvent system comprising water and/or butylene glycol.

For example, the plant may be powdered and mixed in a water/butylene glycol mixture, phase separated, filtered and sterilized. See, e.g., FR2890312 A1.

### Topical Compositions

The Southernwood extract may be combined with one or more cosmetically acceptable carriers to form a composition (e.g., formulation) suitable for use on visibly dry skin.

Suitable carriers of this invention include, but are not limited to, water, as well as diols or polyols, such as those having humectancy properties, including 1,2-propanediol, glycerin, propylene glycol, butylene glycol, polyethylene glycol (PEG), and combinations thereof. While in certain embodiments lower alcohols such as ethanol and isopropanol may be included, in other embodiments, in order to reduce likelihood of sky drying, these alcohols are excluded from the composition (e.g., present in less than 0.5%).

In order to facilitate treatment of visibly dry skin, in certain embodiments of the invention the cosmetically acceptable carrier includes both a humectant as well as an emollient. By emollient it is meant cosmetic ingredients that are generally insoluble in water and suitable for “leave on” applications for the skin.

The emollient may include a hydrophobic moiety that meets one or more of the following three criteria: (a) has a carbon chain of at least six carbons in which none of the six carbons is a carbonyl carbon or has a hydrophilic moiety (defined below) bonded directly to it; (b) has two or more alkyl siloxy groups; or (c) has two or more oxypropylene groups in sequence. The hydrophobic moiety may include linear, cyclic, aromatic, saturated or unsaturated groups. One skilled in the art will recognize that emollients do not include amphiphilic molecules such as emulsifiers, surfactants and other surface active compounds. Amphilphilic molecules that will be understood to be excluded from emollients include those compounds that include both (a) a hydrophobic moiety defined above, and (b) a hydrophilic moiety, such as anionic, cationic, zwitterionic, or nonionic group, that is polar, including sulfate, sulfonate, carboxylate, phosphate, phosphonates, ammonium, including mono-, di-, and trialkylammonium species, pyridinium, imidazolinium, amidinium, poly(ethyleneiminium), ammonioalkylsulfonate, ammonioalkylcarboxylate, amphoacetate, hydroxyl, and poly(ethyleneoxy)sulfonyl. Emulsifiers, surfactants and other surface active compounds are commonly used for emulsification and wetting rather than for film-formation, spreading and the like.

Examples of emollients include mineral oils/waxes, including petrolatum; vegetable oils (glyceryl esters of fatty acids, triglycerides), waxes, other fatty esters. Specific non-limiting examples include, without limitation, isopropyl palmitate, isopropyl myristate, dimethicone, shea butter, petrolatum, C12-C15 alkyl benzoates, caprylic/capric triglycerides, various vegetable waxes, and mineral oil.

Various compounds may be included in the cosmetically-acceptable carrier to alter osmolarity and/or pH to acceptable levels. These include, but are not limited to sodium chloride, sodium phosphate monobasic, sodium phosphate dibasic, sodium hydroxide, and citric acid.

In order to facilitate the formulation of a suitable cosmetically-acceptable carrier, one may include any of various functional ingredients in the composition. For example, one may include any of a number of sequesterants, emulsifiers, thickeners, polymers, preservatives, colorants, fragrances, antioxidants, and other ingredients commonly used in personal care and cosmetic products. Suitable emulsifiers include, for example, non-ionic emulsifiers such as fatty alcohols including polyethoxylated fatty alcohols, esters of glycerol; anionic emulsifiers such as alkyl phosphates; cationic emulsifiers such as alkyl quaternary ammonium compounds. Suitable thickeners include hydrophobically modified acrylic polymers, cellulose polymers and clays.

While in certain embodiments (in which the product is in the form of a cleanser) cleansing surfactants such as betaines, sulfates, sulfonates, polyglycosides, among other wetting agents typically utilized for wetting and foam generation may be included, in other embodiments, in order to reduce likelihood of skin drying and to make the product suitable for a “leave-on” application, cleansing surfactants are excluded from the composition.

The pH chosen is not critical, but may be in a range, for example that is from about 4 to about 8, such as from about 5 to about 7.

The cosmetically acceptable carrier in the topical composition may constitute from about 40% to about 99.99%, by weight, of the composition, more preferably from about 80% to about 95%, by weight, of the composition. In a particularly preferred embodiment, the composition includes at least about 25% by weight water, more preferably at least about 50% by weight water. In other embodiments, the carrier (and the composition) is anhydrous. Such anhydrous compositions may be suitable for, for example, lip treatment products or color cosmetic (e.g., foundation).

In one embodiment, the compositions according to this invention may further contain one or more additional cosmetically active agent(s) as well as the above-mentioned components. What is meant by a “cosmetically active agent” is a compound, which may be a synthetic compound or a compound extracted, isolated, purified or concentrated from a natural source, or a natural extract containing a mixture of compounds, that has a cosmetic or therapeutic effect on the tissue, including, but not limited to: anti-microbial agents such as anti-yeast, anti-fungal, and anti-bacterial agents, anti-inflammatory agents, anti-aging agents, depigmentaion agents, anti-parasite agents, antioxidants, anti-acne agents, keratolytic agents, nutrients, vitamins, minerals, energy enhancers, sunscreens, sebum modulators, anti-cellulite agents and the like.

Examples of vitamins that may be constituents of the compositions of this invention include, but are not limited to, vitamin A, vitamin Bs such as vitamin B3, vitamin B5, vitamin B7 and vitamin B12, vitamin C, vitamin K, vitamin E such as alpha, gamma or delta-tocopherol, and their derivatives (such as salts and esters) and mixtures thereof.

Examples of antioxidants which may be utilized in the compositions and methods of this invention include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), different types of tocopherols (e.g., alpha-, gamma-, and delta-tocopherols and their esters such as acetate) and their mixtures, tocotrienols, and ubiquinone. Natural extracts containing antioxidants suitable for use in the compositions of this invention include, but are not limited to, extracts containing flavinoid, isoflavinoid, and their derivatives such as genistein and diadzein (e.g., such as soy and clover extracts, extracts containing resveratrol and the like. The one or more additional cosmetically active agent(s) may be present in any suitable concentration, such as, for example from about 0.01% to about 10% by weight.

Examples of anti-aging agents that which may be utilized in the compositions and methods of this invention include, but are not limited to, retinoids (e.g., retinol and retinyl palmitate).

Other examples of anti-aging actives include copper containing peptides; vitamins such as vitamin E, vitamin C, vitamin B, and derivatives thereof such as vitamin E acetate, vitamin C palmitate, and the like; antioxidants including beta carotene, alpha hydroxy acids such as glycolic acid, citric acid, lactic acid, malic acid, mandelic acid, ascorbic acid, alpha-hydroxybutyric acid, pyruvic acid; beta hydroxy acids such as beta-hydroxybutyric acid, beta-phenyl-lactic acid, beta-phenylpyruvic acid; polyphenolics; botanical extracts such as green tea, soy products, milk thistle, algae, aloe, angelica, bitter orange, coffee, goldthread, grapefruit, hoellen, honeysuckle, Job's tears, lithospermum, mulberry, peony, puerarua, nice, safflower, and mixtures thereof.

Examples of suitable depigmentation agents include, but are not limited to soy products, retinoids such as retinol; Kojic acid and its derivatives such as, for example, kojic dipalmitate; hydroquinone and it derivatives such as arbutin; transexamic acid; vitamins such as niacin, vitamin C and its derivatives; azelaic acid; placertia; licorice; extracts such as chamomile and green tea, and mixtures thereof, with retinoids, Kojic acid, soy products, and hydroquinone being particularly suitable examples.

Examples of sebum inhibitors include, for example, licorice root extracts, zinc salts such as zinc gluconate, dehydroacetic acid, and glycine derivatives. Examples of anti-cellulite agents include, for example, vasodilators such as green tea, and caffeine.

The Southernwood extract and cosmetically acceptable topical carrier and optional additional cosmetically active agents may be combined in any proportion to form a composition suitable for topical use. In one embodiment of the invention, the topical composition includes at least about 0.1% by weight of the Southernwood extract. In certain embodiments the composition includes at least about 0.5%, and in certain other embodiments from about 0.5% to about 5%, such as from about 1% to about 4%, by weight of the Southernwood extract.

The cosmetically-acceptable carrier and the Southernwood extract may be formulated into any of various product types including, but not limited to solutions, suspensions, emulsions such as microemulsions and nanoemulsions, gels, solids and liposomes. The compositions may be made into a wide variety of cosmetic articles that include but are not limited to lotions, creams, gels, sticks, sprays, ointments, cleansing liquid washes and solid bars, pastes, foams, powders, mousses, wipes, strips, patches, wound dressings and adhesive bandages, hydrogels, film-forming products, as well as facial and skin masks. Other forms can be formulated by those of ordinary skill in the art.

Compositions comprising Southernwood extract may be topically applied to visibly dry or xerotic mammalian skin in order to reduce xerosis present on the face or body, including the hands, feet, scalp, elbows, knees, ankles, nape of the neck, among other areas portions of the skin.

In one embodiment, the compositions of the invention are used to treat mildly xerotic skin. In another embodiment, the compositions are used to treat moderately xerotic skin. In another embodiment, the compositions are used to treat skin having well-defined xerosis.

In certain other embodiments, topical compositions comprising Southernwood extract are topically applied to skin having compromised barrier properties, or skin characterized by certain disease states such as eczematic skin, psoriatic skin, or skin characterized by atopic dermatitis.

The compositions may be applied to the skin in need of such treatment according to a suitable treatment regimen, e.g., every month, every week, every other day, every day, twice a day, or the like.

According to the invention, the composition treats visibly dry skin by, inter alia, increasing the level of ceramides in such skin.

In certain embodiments of the invention, the compositions are applied to visibly dry skin in a manner sufficient to increase the expression of glucosyl ceramide synthase in such skin when tested according to the Gene Expression Test as described in Example 3 in this specification. In one embodiment, the composition is applied in a manner sufficient to increase the expression of glucosyl ceramide synthase after 5 days of treatment by at least 10%, preferably at least 15%, more preferably at least 20%, and most preferably at least 30%, as compared to the skin prior to such treatment.

It is believed that one skilled in the art can, based upon the description herein, utilize the present invention to its fullest extent. The following specific embodiments are to be construed as merely illustrative, and not limitative of the remainder of the disclosure in any way whatsoever. The following non-limiting examples further illustrate the invention.

### EXAMPLES

### Example I

The following composition according to the invention (Example 1 Composition) shown in Table 1 was prepared.

TABLE 1

| | | CONCENTRATION |
|---|---|---|
| INGREDIENT | CHEMICAL NAME | (wt. percent) |
| WATER PHASE | | |
| Deionized Water | Water | 83.95 |
| Ultrez 10 | Carbomer | 0.60 |
| Versene NA | Disodium EDTA | 0.20 |
| OIL PHASE | | |
| Brij 72 | Steareth-2 | 0.75 |
| Brij 721 | Steareth-21 | 1.50 |
| Finsolv TN | C12-15 Alkyl Benzoate | 2.00 |
| POST ADDITIONS | | |
| Dow Corning Q7-9120 | Dimethicone | 5.00 |
| Fluid 20 cst | | |
| Phenonip XB | Phenoxyethanol (and) | 1.00 |
| | Methylparaben (and) | |
| | Ethylparaben (and) | |
| | Propylparaben | |
| Sodium Hydroxide | Sodium Hydroxide | q.s. |
| PULPACTYL | aqueous-glycolic | 5.00 |
| | extract of | |
| | Southernwood | |

The Example 1 Composition was prepared by adding water (Item#1) to a beaker, and the vessel was heated to 55-60° C. Versene and Ultrez 10 were added and the temperature was held at 55-60° C. while it was mixed for 15 minutes or until homogeneous. In a separate vessel, the oil phase ingredients were mixed and held at 55-65° C. The oil phase was slowly added to the water phase. After the phases were completely mixed, the mixture was allowed to cool. When the temperature was below 50° C., dimethicone was added. Phenonip was added when the temperature was below 40° C. The mixture was allowed to mix for 10 minutes. Sodium hydroxide was added to a target pH of 5.4. The mixture was allowed to mix for 10 minutes. PULPACTYL was added, and the mixture was again allowed to mix for 10 minutes.

A Comparative Example 1 Composition was prepared identically to the Example 1 Composition, but without PULPACTYL (Southernwood extract). Additional water was included to compensate for the lack of PULPACTYL.

Example 1 Composition and Comparative Example 1 Composition were tested for their ability to promote healthy skin barrier function using Transepidermal Water Loss (TEWL) analysis on eight human subjects as follows. Measurements were performed on three sites on each leg of each subject. Specifically, the subjects were given both the Example 1 Composition and Comparative Example 1 Composition (placebo). Measurements were performed on (3 sites×8 subjects =) 24 total sites for each leg. Baseline measurements of TEWL, TEWL_{baseline}, were conducted prior to application of product using a closed chamber tool, a VAPOMETER made by Delfin Technologies Ltd., Luopio, Finland. TEWL_{baseline} was determined for each composition by separately averaging the 24 TEWL readings. TEWL_{baseline} for sites which were subsequently treated with the Example 1 Composition was determined to be 9.5, whereas TEWL_{baseline} for sites which were subsequently treated with Comparative Example 1 Composition was determined to be 7.5.

The subjects were instructed to apply one of the compositions consistently to the right leg and the other composition consistently to the left leg. They were asked to repeat this treatment once daily for 2 weeks. After the 2 week period was completed, TEWL was measured again on each leg. Upon averaging, TEWL_{test material} for sites after treatment with the Example 1 Composition was determined to be 7.1. TEWL_{test material} for sites after treatment with the Comparative Example 1 Composition was also determined to be 7.1.

The percent decrease in water loss for each composition was calculated as:

Percent Decrease in TEWL=(TEWL_{baseline}−TEWLₘₐₜₑᵣᵢₐₗ)/TEWL_{baseline}

Percent Decrease in TEWL for the Example 1 Composition according to the invention was determined to be 13%, whereas the Percent Decrease in TEWL for the Comparative Example 1 Composition was determined to be −4%. Based upon the variation in the data, it was determined that the Percent Decrease in TEWL after the treatment with the Example 1 Composition was statistically significant at the 95% confidence level.

### Example II

An Example 2 Composition was prepared identically to Example 1 Composition but with 2% by weight (rather than 5%) of Southernwood extract. Similarly, a Comparative Example 2 Composition was prepared identically to the Example 2 Composition, but without PULPACTYL (Southernwood extract).

The Example 2 Composition and Comparative Example 2 Composition were tested for their ability to promote healthy skin barrier function. Specifically, 44 human female subjects between the ages of 25 and 55 were evaluated for (open chamber) TEWL on the subject's legs, vertically from the ankle bone using a DermaLab Skin Testing Apparatus, available from Cortex Technologies of Hadsund, Denmark. These measurements were recorded as “baseline,” i.e., prior to the application of test products.

In taking the measurements, a hand-held probe was placed on each of two skin surface locations. The probe sampled relative humidity at two points above each surface location, allowing the rate of water loss to be calculated from the measured humidity gradient. Each TEWL measurement was taken over 60 seconds, during which there were 40 seconds of site stabilization followed by 20 seconds during which TEWL was analyzed.

The subjects washed their legs with JOHNSON'S HEAD TO TOE cleanser daily for one week prior to the beginning of the study, as well as during the entire term of the study. Twenty subjects applied the Example 2 Composition to one of their legs and 24 subjects applied the Comparative Example 2 Composition (placebo) to one of their legs. The two compositions were applied two times per day for 3 weeks. After 1 week and after 3 weeks of treatment, TEWL was measured for again for subjects treated with each composition. Furthermore, TEWL was measured 1 week after completing the treatment.

For the whole population of test subjects, the average TEWL_{baseline} was determined to be 7.7 for sites that were subsequently treated with Comparative Example 2 Composition and 10.1 for those that were subsequently treated with the Example 2 Composition.

Following 1 week of treatment, the average TEWL_{test material} was measured to be 7.1 for Comparative Example 2 Composition and 7.3 for Example 2 Composition. Following 3 weeks of treatment, the average TEWL_{test material} was measured to be 6.4 for Comparative Example 2 Composition and 6.8 for Example 2 Composition.

After 3 weeks, no further test material was applied to the subjects. After waiting an additional week, TEWL was measured again to assess the level of retention of the moisture benefits from the previous application. The average TEWL_{test material} measured was 6.5 for Comparative Example 2 Composition and 6.9 for Example 2 Composition.

Percent Decrease in TEWL was calculated (as described in Example I above) for the Comparative Example 2 Composition to be 8%, 17%, and 15% for 1 week, 3 weeks and 4 weeks, respectively. Similarly, Percent Decrease in TEWL for Example 2 Composition was calculated to be 27%, 32%, and 31% for 1 week, 3 weeks and 4 weeks, respectively.

Statistical (T-test) analysis indicated that treatment with the Example 2 Composition provided significantly lower TEWL than for baseline and directionally better Percent Decrease in TEWL as compared with Comparative Example 2 Composition treatment. Furthermore, the results indicate that even one week after treatment was ceased, virtually no regression was observed in test subjects treated with the Example 2 Composition, whereas substantial regression (TEWL values increasing back towards high levels prior to treatment) was observed with the Comparative Example 2 Composition.

### Example III

The following Gene Expression Test was conducted in order to evaluate compositions containing Southernwood extract for their effects in cultured human skin explants. A full thickness skin explant culture (12 mm in diameter), including fat layer, was established using abdominal skins obtained with informed consent from healthy individuals undergoing plastic surgery. Patient identities were not disclosed to preserve confidentiality, in compliance with US HIPAA regulations. Skin explants were either untreated, or treated with 0.5% Southernwood extract (PULPACTYL) in the media. Southernwood extract was diluted with culture media to achieve a final concentration of 0.5%. Culture media, with or without 0.5% Southernwood extract, were changed every day, except during weekends, when a larger volume of media were added. Skin explants with all layers were harvested at indicated time points and stored in Trizol (Invitrogen, Carlsbad Calif.) at −80° C. for RNA extraction.

For Quantitative Polymerase Chain Reaction (QPCR) analysis, total RNA was extracted from skin explants using Trizol 10 (Invitrogen, Carlsbad, Calif.) according to manufacturer's instructions. RNA was then converted to cDNA using Superscript® reverse transcriptase (Invitrogen, Carlsbad, Calif.). QPCR analyses were performed using a 7500 Realtime PCR system (Applied Biosystems, Foster City, Calif.). QPCR reaction, in a 20 μl volume, contained 10 μl QPCR master mix (Applied Biosystems, Foster City, Calif.), 1.5 μl of either forward or reverse primer (5 μM), 5 μl of cDNA, and 2 μl of H2O. Primers used were:

Glucosyl ceramide synthase:
Forward: TGGATCAAGCAGGAGGACTT
reverse: TCCAACCTCGGTCAGCTATC
Caspase 14:
Forward: AAGACAGCCCACAAACCATC
reverse: GAACACATCCACCAGGGTCT
Collagen IV:
Forward: CGCTTACAGCTTTTGGCTCG
reverse: GACGGCGTAGGCTTCTTGAA

QPCR results were normalized to respective day 5 controls to obtain a fold increase in expression levels.

The results from one experiment are shown in Table 2.

TABLE 2

| | Parameters examined | | | |
|---|---|---|---|---|
| | Day 5 | | Day 7 | |
| | Untreated | Southernwood | Untreated | Southernwood |
| | Control | extract 0.5% | control | extract 0.5% |
| Glucosyl | 1 | 1.32 | 1.51 | 13.47 |
| ceramide | | | | |
| synthase | | | | |
| caspase -14 | 1 | 0.43 | 1.89 | 7.65 |
| Collagen IV | 1 | 1 | 0.67 | 3.10 |

Expression of glucosyl ceramide synthase is indicative of ceramide synthesis, caspase 14 is indicative of potential improved skin barrier function, and collagen IV is expressed at the dermal-epidermal junction. Accordingly, the results indicate that Southernwood extract surprisingly up-regulates genes that are involved in ceramide synthesis and healthy barrier function.

It is understood that while the invention has been described in conjunction with the detailed description thereof, that the foregoing description is intended to illustrate and not limit the scope the invention, which is defined by the scope of the appended claims. Other aspects, advantages, and modifications are within the claims.

1. A method of treating visibly dry skin of the body, which comprises topically applying a composition comprising a Southernwood extract to said visibly dry skin of the body.

2. The method of claim 1, wherein said visibly dry skin is xerotic skin.

3. The method of claim 1, wherein said treatment is sufficient to increase the expression of glucosyl ceramide synthase in said skin by at least 10%.

4. The method of claim 1, wherein said skin is characterized by an eczematic disease state.

5. The method of claim 1, wherein said skin is characterized by a psoriatic disease state.

6. The method of claim 1, wherein said Southernwood extract comprises saccharides having a degree of polymerization of 1 to 4 and polyphenols.

7. The method of claim 1, wherein said Southernwood extract comprises monosaccharides, trisaccharides, tetrasaccharides, and polyphenols.

8. The method of claim 1, wherein said composition comprises from about 0.5% to about 5% by weight of said Southernwood extract.

9. The method of claim 1, wherein said composition further comprises a humectant and an emollient.
